(19) United States
(12) Patent Application Publication
(10) Pub. No.: US 2010/0043083 A1
(43) Pub. Date: Feb. 18, 2010

(54) Insect chemosensory receptors and methods of use thereof

(76) Inventors: Leslie B. Vosshall, New York, NY (US); Walton D. Jones, Gyeonggi-do (KR)

Correspondence Address: MEDLEN & CARROLL, LLP, 101 HOWARD STREET, SUITE 350, SAN FRANCISCO, CA, 94105, US

(21) Appl. No.: 11/988,623

(22) PCT Filed: Jul. 14, 2006
(86) PCT No.: PCT/US2006/027433

§ 371 Date: Feb. 17, 2009

Related U.S. Application Data
(60) Provisional application No. 60/698,959, filed on Jul. 14, 2005.

(51) Int. Cl. C12N 15/85; C07K 14/705; C12N 5/10; A01K 67/00

(52) U.S. Cl. 800/21; 530/350; 435/325; 800/13

(57) ABSTRACT

Methods for controlling insect attraction, as well as inhibiting, preventing and reducing the incidence of insect-borne disease in a subject, are described by inhibiting the expression, activity, dimerization or signaling by gustatory receptors that alter insect responsiveness to carbon dioxide. Methods for identifying agents for effecting the same by interfering with expression, activity, dimerization or signaling by gustatory receptors are also provided.

### CROSS-REFERENCE TO RELATED APPLICATION

This application claims priority to U.S. Provisional Patent Application No. 60/698,959, filed Jul. 14, 2006, which is incorporated herein by reference in its entirety.

### STATEMENT AS TO FEDERALLY SPONSORED RESEARCH

This invention was conducted with U.S. Government support under National Institutes of Health grant No. DC005036. The government has certain rights in the invention.

### BACKGROUND OF THE INVENTION

A myriad of diseases are propagated by the insect vector. Malaria, an infectious disease transmitted by female mosquitoes, is one of mankind's most persistent and deadly foes. This disease affects more than 300 million people each year, resulting in greater than one million deaths annually, predominantly of children under the age of five in sub-Saharan Africa. Malaria is more prevalent today in more parts of the world than it was in the 1960s, despite concerted efforts to fight it on many fronts. Climate change due to global warming has been predicted by some to cause a return of malaria to the developed world, which has not seen endemic malaria in a century or longer. The disease is caused by protozoan parasites (Plasmodium falciparum, P. vivax, P. ovale and P. malariae) and is spread to humans by mosquitoes, primarily Anopheles gambiae but including up to 60 different species in the anopheline group.

In the last century, a number of approaches have been put into place to control malaria, and other insect-borne diseases, with mixed results. Chemotherapy agents have been developed that attack various stages of the parasites' life cycles in the human host, however, the development of widespread resistance to these drugs is limiting their usefulness in many countries. Vaccines against the parasites have to date been mostly ineffective.

The role of the insect vector in spreading the disease involves the female mosquito blood meal and delivery of the insect-borne parasite. Insects have a strong attraction to humans, typically biting their hosts at night and indoors when most vulnerable during sleep. Since the discovery of the role of the vector in this disease, efforts have focused on eradicating the vector, including the use of DDT (dichloro-diphenyl-trichloroethane) and other insecticides. Use of DDT as an environmentally applied insecticide has fallen out of favor both because it has unwanted ancillary effects on the viability of other animals and because insect resistance has emerged.

Mosquitoes, and other insects that are attracted to humans, have evolved attraction responses to components of human skin emanations such as carboxylic acids, lactic acid, 1-octen-3-ol, and a variety of other chemicals. They also potently attracted to the carbon dioxide present in human breath.

New methods and particularly agents to control insect-borne diseases which are safe and cost-effective would be particularly useful for these diseases.

### SUMMARY OF THE INVENTION

In one embodiment, this invention provides a method of controlling insect attraction to a subject, the method comprising the step of inhibiting gustatory receptor expression in the insect, whereby inhibiting expression alters insect responsiveness to carbon dioxide, thereby controlling insect attraction to the subject.

In one embodiment, this invention provides a method of controlling insect attraction to a subject, the method comprising the step of inhibiting formation of a heterocomplex of gustatory receptors in the insect, whereby inhibiting formation of the heterocomplex alters insect responsiveness to carbon dioxide, thereby controlling insect attraction to the subject.

In one embodiment, the gustatory receptor is Gr21a, Gr63a, or a homologue thereof, or a combination thereof. In another embodiment, the gustatory receptor is GPRgr22, GPRgr24, or a homologue thereof, or a combination thereof.

In one embodiment, gustatory receptor expression is inhibited via exposure of said insect to an agent, which, in one embodiment is a nucleic acid, or in another embodiment, is a small molecule. In another embodiment, formation of heterocomplexes of gustatory receptors is inhibited by exposure of said insect to an agent. In one embodiment, the agent is a nucleic acid, or in another embodiment, a small molecule.

In another embodiment, this invention provides a method of inhibiting, preventing or reducing the incidence of insect-borne disease in a subject, the method comprising the step of inhibiting gustatory receptor expression in the insect, whereby inhibiting expression alters insect responsiveness to carbon dioxide, controlling insect attraction to the subject, thereby inhibiting, preventing or reducing the incidence of insect-borne disease in a subject.

In another embodiment, this invention provides a method of inhibiting, preventing or reducing the incidence of insect-borne disease in a subject, the method comprising the step of inhibiting formation of a heterocomplex of gustatory receptors in the insect, whereby inhibiting formation of the heterocomplex alters insect responsiveness to carbon dioxide and attraction to the subject, thereby inhibiting, preventing or reducing the incidence of insect-borne disease in a subject.

In one embodiment, the disease is malaria, dengue, yellow fever, river blindness, lymphatic filariasis, sleeping sickness, leishmaniasis, epidemic polyarthritis, West Nile virus disease or Australian encephalitis.

In one embodiment, the method of inhibiting, preventing or reducing the incidence of insect-borne disease is via exposing the insect to an agent, which in one embodiment is a nucleic acid, or in another embodiment is a small molecule. In one embodiment, the agent is applied to a netting.

In another embodiment, modified neurons are provided that are engineered to express at least one gustatory receptor, for example, Gr21a, Gr63a, GPRgr22, GPRgr24, or a homologue thereof, or any combination thereof. In one embodiment, the modified neurons are ab3A neurons. In another embodiment, modified neurons are provided that are express Gr21a and Gr63a, or homologues thereof, or express GPRgr22 and GPRgr24, or homologues thereof.

In another embodiment, this invention provides an agent, which inhibits gustatory receptor expression or function. In another embodiment, this invention provides an agent, which inhibits gustatory receptor heterocomplex formation or trafficking of the complex to ciliated dendrites. In another embodiment, the gustatory receptor heterocomplex comprises Gr21a and Gr63a, or homologues thereof. In yet another embodiment, the gustatory receptor heterocomplex comprises GPRgr22, GPRgr24, or homologues thereof.

In another embodiment, this invention provides an isolated protein complex comprising insect gustatory receptors Gr21a and Gr63a, or homologues thereof. In still another embodiment, this invention provides an isolated protein complex comprising insect gustatory receptors GPRgr22, GPRgr24, or homologues thereof.

In another embodiment, screening methods are provided for identifying agents that modulate the expression or trafficking of one or more gustatory receptors. In another embodiment, screening methods are provided for identifying agents that modulate the formation of a heterodimer between a first gustatory receptor and a second gustatory receptor.

In other embodiments mutant flies which are Gr21a null or Gr63a null are provided.

### BRIEF DESCRIPTION OF THE DRAWINGS

FIG. 1 shows fluorescent double in situ hybridization using an anti-Gr21a-FITC probe and either anti-Gr63a-DIG (FIG. 1A) or anti-Gr10a-DIG (FIG. 1C). Sequential TSA amplifications using TSA-FITC and TSA-Cy5 kits (Perkin Elmer) with peroxidase quenching between reactions allowed visualization of weak signals. Gr21a and Gr63a are co-expressed, whereas Gr21a and Gr10a are expressed in non-overlapping cell populations. FIG. 1B demonstrates the visualization of antennal lobe projections of Gr21a neurons and Gr63a neurons onto the V glomerulus. Transgenic fly (Gr63a-syntRFP;Gr21a-GAL4;UAS-CD8-GFP) brains were dissected and labeled with an antibody recognizing central brain neuropil (nc82), and visualized with a secondary antibody coupled to Cy5. nc82 staining is in blue. The synapses of Gr63a neurons are labeled with the Gr63a promoter fusion to synaptotagmin-RFP and are in magenta. Gr21a neuronal membranes are labeled green with the membrane marker CD8-GFP under control of a Gr21a-GAL4 driver. The overlap in the V glomerulus appears white. Anopheles gambiae, GPRgr22 and GPRgr24, the homologues of Drosophila Gr21a and Gr63a, are co-expressed in the maxillary palp (FIG. 1D). GPRgr22 and GPRgr24 are highly conserved from flies to mosquitoes (FIG. 1E). Percent identity values are given in blue. GPRgr22 is not co-expressed with the mosquito orthologue of the broadly-expressed fly co-receptor Or83b (FIG. 1F).

FIG. 2 shows CO₂ sensitivity from the expression of various combinations of GRs in the ab3A neuron. Only the combination of Gr21a and Gr63a (last row) confers significant CO₂ responsivity on this neuron that normally responds strongly to ethyl hexanoate. Sample traces show 4 seconds of activity surrounding a 1 second stimulus of either room air (0.03% CO₂) or roughly 3% CO₂. Spikes were counted in a 500 ms window immediately following the stimulus. 2(CO₂−Air) gives a corrected CO₂ response in spikes/sec. N=12-18 sensilla/genotype.

FIG. 3A-B shows that Gr63a null mutant flies created by homologous recombination fail to respond to CO₂, demonstrating that Gr63a is an essential component of a CO₂ receptor in Drosophila. FIG. 3A shows the targeting construct (top), schematic of site of homologous recombination (middle), and mutant chromosome (bottom). The flanking genes included in the targeting construct are not disrupted by this manipulation. FIG. 3B shows representative traces from wild type (top) and Gr63a−/− (bottom) ab1 sensilla.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This invention provides, in some embodiments, methods for controlling insect attraction to a subject, and methods for inhibiting, preventing and reducing the incidence of insect-borne disease in a subject, and agents for effecting the same.

One approach to controlling insect-borne diseases in man is to disrupt the interaction between the insect vector and its human host, and this approach represents an embodiment of this invention. Many insects rely primarily on their sense of smell to find humans by cuing in on, inter-alia, the carbon dioxide present in breath.

Mosquitoes, for example, sense odors through olfactory neurons located on their antennae and maxillary palps, functionally analogous to the same structures in the fruit fly, Drosophila melanogaster. These olfactory appendages are covered with hundreds of porous sensory hairs, called sensilla, which house the olfactory sensory neurons (OSNs), which recognize odorants and transmit odor-specific information to the brain. Odorant receptors are membrane proteins that reside in the sensory dendrite of the OSN, which bind odors, and transduce odor-binding to neuronal activation. Odorant receptor (OR) genes have been described in insects, such as Drosophila, whose genome contains approximately 60 OR genes, each encoding a different seven transmembrane domain receptor protein. A second gene family of gustatory receptors (GRs), primarily involved in taste, has been described, with some of its members expressed in the olfactory system and having olfactory function.

As exemplified herein, insects expressing Gr21a and Gr63a, showed robust responses to carbon dioxide (CO₂) in their basiconic sensilla, with Gr63a null mutants not responding to CO₂.

In another embodiment, the same co-expression of the CO₂ receptor subunits is identified in the malaria mosquito, Anopheles gambiae. The Gr21a and Gr63a orthologues, GPRgr22 and GPRgr24, are co-expressed in the CO₂-sensitive olfactory organ in the mosquito, the maxillary palp. As in Drosophila, the putative CO₂ receptor is not co-expressed with the mosquito orthologue of the ubiquitous olfactory co-receptor of the Or83b subtype (GPRor7 in the mosquito).

In one embodiment, this invention provides a method of controlling insect attraction to a subject, the method comprising the step of inhibiting gustatory receptor expression in the insect, whereby inhibiting expression alters insect responsiveness to carbon dioxide, thereby controlling insect attraction to the subject.

In one embodiment, the term “insect” is used herein in its broad common usage to include spiders, mites, ticks and like pests which are not in the strict biological sense classified as insects, yet possess gustatory receptors, which are involved in CO₂ sensation in the insect. In one embodiment, the term “insect” is to conform to the definitions provided by Congress in Public Law 104, the “Federal Insecticide, Fungicide, and Rodenticide Act” of 1947, Section 2, subsection h, wherein the term “insect” refers not only to those small invertebrate animals belonging mostly to the class Insecta, comprising six-legged usually winged forms, as beetles, bugs, bees, flies, and so forth, but also to other allied classes of arthopods whose members are wingless and usually have more than six legs, as spiders, mites, ticks, centipedes, and wood lice. As will be appreciated by one skilled in the art, many arthropods, aside from winged members, carry pathogens which when in contact with a subject, cause disease. It is to be understood that the methods, agents, and compositions of this invention may be applied for use with any member of what is broadly encompassed by the term “insect”, as described herein, and is to be considered as an embodiment thereof.

In one embodiment, the term “controlling insect attraction” refers to both enhancing, stimulating or in any way positively affecting insect attraction to a subject. In one embodiment, the term “controlling insect attraction” refers to diminishing, abrogating or in any way negatively affecting insect attraction to a subject.

In one embodiment, the term “controlling insect attraction” may be ascertained as a measure of downstream effects of insect attraction to a subject, such as, in terms of the number of bites, transmission of carried material, including pathogens carried within the insect. In one embodiment, controlling insect attraction may be reflected in terms of occupancy of a region of space proximal to a subject, frequency of crossing a region of space, etc. In another embodiment, the subject may be behind a netting and insect attraction to the subject may be affected by the frequency of insect approximation to the netting. In one embodiment, the netting may be adapted such that insect approach within a specified proximity to the netting may be sufficient to trap the insect. In another embodiment, an electrified region may separate the subject from the insect, such that insect approach within a specified proximity to the netting may be sufficient to electrocute or stun the insect. Manipulation of these conditions, in turn, represents other embodiments of the methods of this invention, as will be appreciated by one skilled in the art, and as is discussed further herein.

In one embodiment, inhibiting gustatory receptor expression in the insect affects insect attraction, positively or negatively, as a function of the insect species. For example, CO₂ is the most potent repellent known for fruit flies, while the same stimulus is a potent attractant for mosquitoes. As exemplified herein, blockade of neurons expressing the gustatory receptors, Gr21a and Gr63a, was sufficient to completely block repulsion to CO₂ in Drosophila. As further exemplified herein, disruption of the single gustatory receptor Gr63a abolishes electrical responses to CO₂ in the Drosophila antenna. Since Anopheles exhibit the opposite behavior, in one embodiment, blockade of the Gr21a orthologue GPRgr22 or the Gr63a orthologue GPRgr24 is sufficient to completely block attraction to CO₂ in mosquitoes. It will be appreciated by the skilled artisan, that blockade or abrogation of expression of particular gustatory receptors may be accomplished via means well known in the art, with CO₂ responsiveness readily assesses as a function of the introduced changes in expression, via assays as described and exemplified herein, and others known to those of skill in the art, any of which may be used in accordance with the methods and agents of this invention, and are to be considered embodiments thereof.

In one embodiment, the method involves inhibiting gustatory receptor expression, wherein the receptor is associated with or involved in CO₂ sensation. In one embodiment, the gustatory receptor or receptors comprise Gr21a, Gr63a, or homologues thereof, of a combination thereof. In another embodiment the gustatory receptor or receptors comprise GPRgr22, GPRgr24, or homologues thereof, or a combination thereof. In another embodiment, orthologues of the aforementioned gustatory receptors, homologues thereof and heterocomplexes thereof are embraced in other CO₂ sensitive insect species.

In one embodiment, the term “homologue” refers to a molecule which shares sequence identity or functional comparability to the reference molecule. In one embodiment, the term “homologue” encompasses a molecule with at least 65% correspondence with the indicated molecule, in terms of, in one embodiment, its structure, or in another embodiment, amino acid sequence, or in another embodiment, nucleic acid sequence, or in another embodiment function. In another embodiment, the molecule exhibits at least 70% correspondence with the indicated sequence or structure. In another embodiment, the molecule exhibits at least 72% correspondence with the indicated sequence or structure. In another embodiment, the molecule exhibits at least 75% correspondence with the indicated sequence or structure. In another embodiment, the molecule exhibits at least 80% correspondence with the indicated sequence or structure. In another embodiment, the molecule exhibits at least 82% correspondence with the indicated sequence or structure. In another embodiment, the molecule exhibits at least 85% correspondence with the indicated sequence or structure. In another embodiment, the molecule exhibits at least 87% correspondence with the indicated sequence or structure. In another embodiment, the molecule exhibits at least 90% correspondence with the indicated sequence or structure. In another embodiment, the molecule exhibits at least 92% correspondence with the indicated sequence or structure. In another embodiment, the molecule exhibits at least 95% or more correspondence with the indicated sequence or structure. In another embodiment, the molecule exhibits at least 97% correspondence with the indicated sequence or structure. In another embodiment, the molecule exhibits at least 99% correspondence with the indicated sequence or structure. In another embodiment, the molecule exhibits 95%-100% correspondence with the indicated sequence or structure. Similarly, as used herein, the reference to a correspondence to a particular molecule includes both direct correspondence, as well as homology to that molecule as herein defined. In one embodiment, the homologue will share 48% identity with the reference sequence, or in another embodiment, the homologue will share 59% similarity with the reference sequence, for example Gr63a and GPRgr24 share 48% identity and 59% similarity. In another embodiment, the homologue will share 62% identity with the reference sequence, or in another embodiment, the homologue will share 75% similarity with the reference sequence, for example Gr21a and GPRgr22 share 62% identity and 75% similarity.

Sequence homology or identity at the amino acid or nucleotide level may be determined, in some embodiments, by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases (see Altschul et al., (1994) Nature Genetics 6, 119-129 which is fully incorporated by reference). The search parameters for histogram, descriptions, alignments, expect (i.e., the statistical significance threshold for reporting matches against database sequences), cutoff, matrix and filter may be at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference). For blastn, the scoring matrix may be set by the ratios of M (i.e., the reward score for a pair of matching residues) to N (i.e., the penalty score for mismatching residues), wherein the default values for M and N are 5 and −4, respectively.

The term “homology”, or “homologue” may thus refer to a molecule sharing sequence identity, structural identity, or functional identity to the reference sequence. The term “homologue” is to be understood to encompass an “orthologue” or “paralogue”, where the molecule is involved in carbon dioxide sensation.

By using the term “homology” and other like forms, it is to be understood that any molecule, that functions similarly, and/or contains sequence identity, and/or is conserved structurally so that it approximates the reference molecule, is to be considered as part of this invention.

In one embodiment, the gustatory receptor comprises Gr21a, or a homologue thereof. In one embodiment, the Gr21a receptor is encoded by transcript GR21D.1, which has a sequence as described in NCBI's genbank, having an accession number of AC004420, range 34784-33509. In one embodiment, Gr21a is as described in U.S. Patent Application Publication Number 20040003419.

In one embodiment, Gr63a refers to the Gr63F1 protein described in U.S. Patent Application Publication Number 20030045472.

In another embodiment, the homologue is a gustatory receptor from an insect of the Anopheles genus, and the homologue may comprise GPRgr22/GPRgr24, or a homologue thereof.

In one embodiment, the GPRgr22 amino acid sequence for use in the methods of this invention, for example in generating/designing the agents of this invention may correspond to, or be homologous to the following:

>gi|31234894|ref|XP_319142.1| ENSANGP00000011853 [Anopheles ganbiae str. PEST]
MIHTQMEDAAYEIRHQVLNPNQRQQLEDRRRIKEQLHQLEQDNESPTHMYRRKLKIASDVNLLDQHDSFY
HTTKSLLVLFQIMGVMPIMRSPKGVDMPRTTFTWCSKAFLWAYFIYACETVIVLVVARERINKFISTSDK
RFDEVIYNIIFMSIMVPHFLLPVASWRNGSEVAKFKNMWTDFQYKYLIVTGKPIVFPKLYPITWTLCIVS
WSLSLVIILSQYYLQPDFQFCHTFAYYHIIAMLNGFCSLWFVNCTAFGTASKAFAKELTDVLATERPAAK
LTEYRHLWVDLSHMMQQLGKAYSNMYGIYCLVIFFTTIIATYGSLSEIIEHGATYKEVGLFVIVFYCMSL
LFIICNEAHHASKRVGLNFQERLLNVNLTAVDKATQKEVEMFLVAIDKNPPTMNLDGYANINRGLITSNI
SFMATYLVVLMQFKLTLLRQSAKNAFISALKANLSRIRSLDADKVNT (SEQ ID NO: 1).

In one embodiment, the GPRgr22 nucleic acid sequence for use in the methods of this invention, for example in generating/designing the agents of this invention may correspond to, or be homologous to the following:

| | |
|---|---|
| 1 | atgattcaca cacagatgga agatgctcag tacgagattc gccatcaggt gctgaatccg |
| 61 | aatcaacgtc agcagctgga ggacaggcgc cgcatcaagg agcaactgca tcagctcgaa |
| 121 | caggataacg agtctccgac gcacatgtac aggcgtaagc tgaagattgc tagcgatgta |
| 181 | aacctgctcg accagcacga ttcgttctac cacaccacca agagtctgct ggtgctgttc |
| 241 | caaattatgg gcgtgatgcc gataatgcgc agcccgaaag gtgtcgacat gccccgtacc |
| 301 | acgtttacct ggtgctccaa ggcgtttctc tgggcgtact ttatctacgc ctgcgaaacg |
| 361 | gtcattgtgc tggtggtggc ccgggagcgc attaacaaat tcatctcaac cagcgacaag |
| 421 | cggttcgatg aggtgatcta caacatcatc tttatgtcga taatggtgcc ccattttctg |
| 481 | cttcccgtag caagctggcg taacggttcg gaggtggcaa aattcaaaaa catgtggaca |
| 541 | gattttcagt acaagtatct catcgtcacc ggaaagccca tcgtttttcc gaagctgtat |
| 601 | ccgatcacgt ggaccttgtg catcgtatcg tggtcgttga gtttggtgat catcctgtcg |
| 661 | caatattacc tgcagccaga ctttcagttc tgtcacacct ttgcctacta ccacatcatt |
| 721 | gcaatgttga acgggttctg tagcctttgg ttcgtcaact gcaccgcatt tgggacggct |
| 781 | agtaaggcct ttgccaaaga actaacagac gtactggcga ccgaacggcc agccgcgaaa |
| 841 | ctgaccgaat atcgtcacct gtgggtggat ttaagccata tgatgcaaca actgggtaaa |
| 901 | gcatattcca acatgtacgg catctactgt ttggtgatat tcttcaccac tattatcgcc |
| 961 | acctacggct cgctgagcga aattatcgag cacggtgcca cgtataagga agtgggttta |
| 1021 | tttgtcattg ttttctattg tatgagcttg ctgttcatca tttgcaacga agctcatcac |
| 1081 | gcatccaaaa gggtcggatt gaactttcag gaacgtttgt tgaatgtaaa tttaacggcg |
| 1141 | gtggacaagg ctacacaaaa ggaggtagaa atgtttctgg ttgccatcga caaaaatccc |
| 1201 | cctacgatga atcttgacgg gtacgccaac atcaatcgcg gactcatcac atcgaacata |
| 1261 | tccttcatgg cgacgtattt ggtcgttttg atgcagttca aattgacctt actacgacaa |
| 1321 | agtgctaaaa atgctttcat ttctgcactc aaggctaact tgtctagaat tcgatcgctt |
| 1381 | gacgcagaca aagtgaacac ctaa (SEQ ID NO: 2) |

In one embodiment, the GPRgr24 amino acid sequence for use in the methods of this invention, for example in generating/designing the agents of this invention may correspond to, or be homologous to the following:

>gi|58395311|ref|XP_321150.2| ENSANGP00000020131 [Anopheles gambiae str. PEST]
MVFESSKPIYLVLRAIGVLPYTRLPSGGTAFVLASPSMTYCVLFELLLTVYIAFILLNRIEIVRTLEGRF
EESVIAYLFIVNILPILIIPLMWYESRKVVSVVNGWVDFETVYRETTGRALELRLRTKAQVIAILLPILC
SLSVAITHVTMVDFKLLQVIPYCVLDTITYMMGGYWYMACETLSITAKILAEDFQRALRHVGPAAKVSEY
RSLWLRLSKLARDTGFSTCYTFTFICLYLFFIITLSIYGLMSQISDGFGVKDIGLAVTAFCSVGLLFYIC
DEAHYASFNVRTNFQKKLLMVELSWMNTDAQTEINMFLRATEMNPSSINLGGFFDVNRTLFKSLLATMVT
YLVVLLQFQISIPDEPSAMLMHSNSSHA (SEQ ID NO: 3).

In one embodiment, the GPRgr24 nucleic acid sequence for use in the methods of this invention, for example in generating/designing the agents of this invention may correspond to, or be homologous to the following:

| | |
|---|---|
| 1 | atggtgtttg aaagctccaa accgatctac ctggtgctgc gtgccatcgg tgtgctgccg |
| 63 | tacacgcggc tcccgtcggg cggaaccgct tttgtccttg cctccccgtc gatgacgtac |
| 121 | tgtgtgctgt ttttcetgct gctcaccgtg tacattgcct tcatcctgct caatcgcatc |
| 181 | gagatcgtgc gcacgctcga gggccgcttc gaggagtccg tcattgcgta cctgttcatc |
| 241 | gtgaacattc tgccgatcct gatcataccg ctgatgtggt acgaatcgcg caaggtcgtg |
| 301 | tcggtcgtga atgggtgggt cgattttgaa accgtctacc gggaaacaac gggccgggcg |
| 361 | ctggagttgc gcctgcgcac caaagcgcag gtcatagcga tcctgctgcc gatcctgtgc |
| 421 | tccctctcgg tcgccatcac gcacgtgacg atggtggact tcaagctgot gcaggtcatc |
| 481 | ccgtactgtg tgctcgacac gatcacctac atgatgggcg ggtactggta tatggcctgt |
| 541 | gagacgctca gcattaccgc gaaaatactg gcggaagact ttcagcgcgc gctacggcac |
| 601 | gtcggcccgg cggcgaaggt gtccgagtat cggtcgctat ggttacggct gagcaagctg |
| 661 | gcacgggata cgggcttttc cacctgctac acgttcacgt tcatctgtct gtatctgttc |
| 721 | ttcatcataa cgctttcgat ctatgggctg atgtcgcaaa tttcggacgg cttcggcgtg |
| 781 | aaggacatcg ggctggcggt gaccgccttc tgtagcgtgg ggctgctgtt ctacatctgc |
| 841 | gacgaggcgc actacgcgtc gttcaatgtg cgcaccaact tccagaagaa gctgctgatg |
| 901 | gtggagctga gctggatgaa cacggacgcg cagacggaga ttaatatgtt tctgogtgcg |
| 961 | accgagatga atccttccag catcaatctt ggcgggtttt tcgacgttaa tcgcacgctg |
| 1021 | tttaagtcgc tgcttgccac gatggttacg tatctggtgg tgttgctaca gttccagatc |
| 1081 | agtattcccg atgagcccag cgccatgctg atgcacagta attcgtctca ttcttag |
| | (SEQ ID NO: 4). |

In another embodiment of this invention, homologues, in particular, in some embodiments, orthologues of gustatory receptors involved in CO₂ sensation may be identified. Such identification may be accomplished via means well known in the art. For example, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules. For example, degenerate primers can be used to clone a particular Drosophila Gustatory Receptor (DGR) gene across species. Specifically, based on the sequence information derived from the family of Drosophila Gustatory Receptors, degenerate primers can be designed based on conserved sequences among gustatory receptors, which can then be used to clone nucleic acid molecules encoding gustatory receptor proteins from other species of insects. In one embodiment, primers are designed, by means well known in the art for specifically cloning the desired GR orthologue, or in another embodiment, multiple gustatory receptors are cloned, and the translated products are screened for functional activity in CO₂ sensation, via methods exemplified and described herein.

In one embodiment, modified neurons are provided that are engineered to express at least one gustatory receptor, such neurons not normally expressing such receptor. In one embodiment, the modified neurons are ab3A neurons. In another embodiment, a modified neuron is provided that expresses Gr10a. In another embodiment, a modified neuron is provided that expresses Gr21a. In another embodiment, a modified neuron is provided that expresses Gr63a. In another embodiment, a modified neuron is provided that expresses both Gr10a and Gr21a. In another embodiment, a modified neuron is provided that expresses both Gr10a and Gr63a. In another embodiment, a modified neuron is provided that expresses both Gr21a and Gr63a. In another embodiment, a modified neuron is provided that expresses GPRgr21. In another embodiment, a modified neuron is provided that expresses GPRgr24. In another embodiment, a modified neuron is provided that expresses both GPRgr21 and GPRgr24. In the foregoing embodiments, the neuron can be a member of the ab3A class of neurons.

In one embodiment, gustatory receptor expression is inhibited via exposure of the insect to an agent. The agent, in turn, represents another embodiment of this invention.

In one embodiment, gustatory receptor expression may be inhibited via any means known in the art. For example, and in some embodiments, gustatory receptor expression may be inhibited via known means of abrogating or diminishing gene expression, wherein the gene encodes the particular gustatory receptor.

In some embodiments, inhibition of expression may be accomplished via the use of RNAi, antisense oligonucleotides, ribozymes, or, in other embodiments, inhibition may be accomplished through known gene knockout techniques. In other embodiments, peptides or small molecules may be used. Agents for effecting the inhibition, in turn, represent other embodiments of this invention.

RNAi Double-stranded RNA directs the sequence-specific degradation of mRNA through RNA interference. The process is known to occur naturally in a wide variety of organisms, including embryos of mammals and other vertebrates, or can be accomplished via introduction of small interfering RNA duplexes ranging in length of between 20-25 nucleotides (US Patent Application No. 20020086356A1). Sequence specific duplex RNAi mediates cleavage of the corresponding mRNA, and therefore provides a useful tool for in vivo degradation of mRNA prior to translation, hence inactivation of protein expression.

The RNAi typically mediating mRNA degradation comprises duplex or double-stranded RNA, but may also include single-stranded RNA, isolated RNA (partially purified RNA, essentially pure RNA, synthetic RNA, recombinantly produced RNA), as well as altered RNA that differs from naturally occurring RNA by the addition, deletion, substitution and/or alteration of one or more nucleotides. Such alterations can include addition of non-nucleotide material, such as to the end(s) of the 20-25 nt RNA or internally (at one or more nucleotides of the RNA) and may also comprise non-standard nucleotides, including non-naturally occurring nucleotides or deoxyribonucleotides.

Antisense oligonucleotides Antisense oligonucleotides represent another known means of inhibiting gene expression, which may be used for the methods of the present invention, representing an embodiment thereof. Antisense oligonucleotides are chimeric molecules, containing two or more chemically distinct regions, each made up of at least one nucleotide. These chimeric oligonucleotides typically contain at least one region wherein the oligonucleotide is modified so as to confer upon the oligonucleotide an increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids, which according to this aspect of the invention, serves as a means of gene silencing via degradation of specific sequences. Cleavage of the RNA target can be routinely detected by gel electrophoresis or in other embodiments, associated nucleic acid hybridization techniques known in the art.

The chimeric antisense oligonucleotides may, in one embodiment, be formed as composite structures of two or more oligonucleotides and/or modified oligonucleotides, as is well described in the art (see, for example, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922), and can, in another embodiment, comprise a ribozyme sequence.

Antisense molecules can be delivered exogenously or by contacting a cell or cells of the insect with a vector capable of directing transcription of an antisense RNA molecule. Baculovirus vectors have been employed for the production of antisense RNA in insects (e.g., Vlak and Chejanovsky, The XIVth International Plant Protection Congress Meeting Report, page 15, 1999), and such technology may be readily adapted herein for delivery of the desired agent for inhibiting gustatory receptor expression, for example, for use in any of the methods of this invention.

Ribozymes Ribozymes may be used for the sequence-specific inhibition of gene expression by the cleavage of mRNAs encoding proteins of interest [Welch et al., “Expression of ribozymes in gene transfer systems to modulate target RNA levels.” Curr Opin Biotechnol. 1998 October; 9(5):486-96]. The possibility of designing ribozymes to cleave any specific target RNA has rendered them valuable tools in both basic research and therapeutic applications [Welch et al., “Ribozyme gene therapy for hepatitis C virus infection.” Clin Diagn Virol. 1998 Jul. 15; 10(2-3):163-71.].

Gene Knockout Techniques In another embodiment, expression can be inhibited/downregulated by “knocking out” the gene encoding a gustatory receptor. Typically this is accomplished by disrupting the gene, the promoter regulating the gene or sequences between the promoter and the gene. Such disruption can be specifically directed by homologous recombination where a “knockout construct” contains flanking sequences complementary to the domain to which the construct is targeted. Insertion of the knockout construct (e.g. into the gustatory receptor encoding gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon).

In other embodiments of the invention, flies in which the gene encoding Gr21a or Gr63a is knocked out are provided, thus providing Gr21a null or Gr63a null flies.

In another embodiment, gustatory receptor expression may be inhibited via other means, such as, for example, via the use of an antibody. For example, and in some embodiments, gustatory receptor expression may be inhibited via known means of abrogating or diminishing gene expression, wherein the gene encodes the particular gustatory receptor.

It is to be understood that the methods of gene regulation as described above involve nucleic acid delivery to the insects, which may be accomplished via delivery of the nucleic acids in their native form, or, in another embodiment within an expression vector, for example, a self-replicating plasmid, or in another embodiment, a viral vector, for example a retroviral vector, as will be appreciated by one skilled in the art.

Methods for introducing the nucleic acids are well known to those skilled in the art, and may include, in some embodiments, the use of lipid or liposome based delivery (WO 96/18372; WO 93/24640; Mannino and Gould-Fogerite (1988) BioTechniques 6(7): 682-691; Rose U.S. Pat. No. 5,279,833; WO 91/06309; and Feigner et al. (1987) Proc. Natl. Acad. Sci. USA 84: 7413-7414) or the use of replication-defective retroviral vectors comprising a nucleic acid sequence as described (see, e.g., Miller et al. (1990) Mol. Cell. Biol. 10:4239 (1990); Kolberg (1992) J. NIH Res. 4: 43, and Cometta et al. (1991) Hum. Gene Ther. 2: 215) Anderson, Science (1992) 256: 808-813; Nabel and Feigner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 11491154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Bohm (eds) Springer-Verlag, Heidelberg Germany; and Yu et al., (1994) Gene Therapy, 1: 13-26.

In other embodiments, modified neurons are provided that are engineered to express at least one gustatory receptor, for example, Gr21a, Gr63a, GPRgr22, GPRgr24, or a homologue of any of the foregoing, or any combination of any of the foregoing. The neurons did not express the at least one gustatory receptor prior to modification. In one embodiment, the modified neurons are ab3A neurons. In another embodiment, modified neurons are provided that express Gr21a and Gr63a, or a homologue of any of the foregoing, or express GPRgr22 and GPRgr24, or a homologue of any of the foregoing. In another embodiment, a neuron expressing at least one gustatory receptor is modified to express a second gustatory receptor. In another embodiment, the second gustatory receptor forms a heterocomplex with the at least one gustatory receptor.

In another embodiment, the methods may be effected by any agent that inhibits gustatory receptor expression or function. In one embodiment, the term “gustatory receptor expression” refers to transcript expression, protein expression, and modifications thereto. In one embodiment, controlling receptor expression will affect receptor function by virtue of absent or altered proteins, which no do not function identically as compared to receptors not subjected to the methods of this invention.

Agents which may be used in any of the methods of this invention, in some embodiments, are randomly selected or rationally selected or designed. In one embodiment, the term “randomly selected” refers to when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

According to this aspect of the invention, and in one embodiment, the agent's activity may then be evaluated, via the assays and methods described herein, and as will be understood by one skilled in the art. For example, and in one embodiment, an agent which inhibits insect attraction, which may function via inhibiting gustatory receptor expression, may be screened by contacting an insect or population with the agent, and evaluating receptor expression following such contact, in one embodiment, or, in another embodiment, via evaluating behavioral responses to CO₂. Similarly, agents which inhibit insect attraction, via preventing an association between two gustatory receptors, as described herein, may be functionally evaluated via determining the presence and/or quality of the association between the two gustatory receptors, via, for example, gel electrophoresis assay, or in another embodiment, a FRET assay may be used to demonstrate proximity of the labeled proteins, which may be conducted in cellular systems, and others, as will be appreciated by one skilled in the art.

Any means whereby the desired function of the agent may be assessed may be utilized in turn, in screening randomly generated agents, and is to be considered as part of this invention.

In one embodiment, an agent is said to be rationally selected or designed when the agent is chosen on a non-random basis which takes into account the sequence of the target site and its conformation in connection with the agent's action. Agents can be rationally selected or rationally designed by utilizing the peptide sequences to identify proposed binding motifs, glycosylation and phosphorylation sites on the protein.

The agents of the present invention can be, as examples, peptides, small molecules, etc. A skilled artisan can readily recognize that there is no limit as to the structural nature of the agents of the present invention. Dominant-negative proteins, DNA encoding these proteins, antibodies to these proteins, peptide fragments of these proteins or mimics of these proteins may be contacted with cells to affect function. “Mimic” as used herein refers to the modification of a region or several regions of a peptide molecule to provide a structure chemically different from the parent peptide but topographically and functionally similar to the parent peptide (see Meyers, (1995) Molecular Biology & Biotechnology, VCH Publishers).

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included. In another embodiment, the agent may be generated synthetically, for example, if a nucleic acid, via translation of sequences subjected to any mutagenesis technique, as well as via protein evolution techniques, well known to those skilled in the art.

Another class of agents of the present invention comprises antibodies immunoreactive with critical positions of proteins or complexes of, and in use in the methods of the invention. Antibody agents are obtained by immunization of suitable mammalian subjects with peptides, containing as antigenic regions, those portions of the protein intended to be targeted by the antibodies.

Antibodies directed to an insect gustatory receptor may be serum-derived or monoclonal and are prepared using methods well known in the art. For example, monoclonal antibodies are prepared using hybridoma technology by fusing antibody producing B cells from immunized animals with myeloma cells and selecting the resulting hybridoma cell line producing the desired antibody. Cells, which express the receptor may be used as immunogens to raise such an antibody. Alternatively, synthetic peptides may be prepared using commercially available machines.

As a still further alternative, DNA, such as a cDNA or a fragment thereof, encoding the receptor or a portion of the receptor may be cloned and expressed. The expressed polypeptide may be recovered and used as an immunogen.

The resulting antibodies are useful to inhibit the function of the gustatory receptor, and may also be useful for identifying or isolating other insect gustatory receptors involved in CO₂ sensation. For example, antibodies against the Drosophila gustatory receptor may be used to screen a tick or sandfly expression library for a tick or sandfly gustatory receptor. Such antibodies may be monoclonal or monospecific polyclonal antibody against a selected insect gustatory receptor. Different insect expression libraries are readily available and may be made using technologies well-known in the art.

In another embodiment, designing of and obtaining the agent may be facilitated via use of an isolated protein complex comprising insect gustatory receptors Gr21a and Gr63a, or homologues thereof, or GPRgr22, GPRgr24, or homologues thereof. Accordingly, in one embodiment of this invention, there is provided a protein complex comprising insect gustatory receptors Gr21a and Gr63a, or homologues thereof. In another embodiment, there is provided a protein complex comprising insect gustatory receptors GPRgr22 and GPRgr24, or homologues thereof. Such protein complexes may be crystallized, by methods well known in the art. Once crystallized, structural information may be readily obtained, by known methods, including, inter-alia, x-ray diffraction analysis and NMR. Values obtained may processed scaled, and modeled using various computer software, for example, DENZO/SCALEPACK (Otwinowski, Z. & Minor, M. in Macromolecular Crystallography, part A, 307-326 (Academic Press (New York), 1997) an electron density map may be obtained, for example, using MLPHARE followed by density modification and phase combination by DM (Collaborative Computational Project, N. The CCP4 suite: programs for protein crystallography. Acta Crystallogr D Biol Crystallogr 50, 760-3 (1994)). Iterative model building and refinement may be performed, for example using the programs O (Jones, T. A., Zou, J. Y., Cowan, S. W. & Kjeldgaard. Improved methods for building protein models in electron density maps and the location of errors in these models. Acta Crystallogr A 47 (Pt 2), 110-9 (1991)) and CNS (Brunger, A. T. et al. Crystallography & NMR system: A new software suite for macromolecular structure determination. Acta Crystallogr D Biol Crystallogr 54 (Pt 5), 905-21 (1998)). Secondary structures may be assigned using, for example, the program DSSP (Kabsch W., a S., C. Biopolymers 22, 2577-2637 (1983)). Graphics may be generated using the programs PyMOL (DeLano, W. L. (http://www.pymol.org, 2002)), and sequence alignments may be performed using ClustalX (Thompson, J. D., et al. Nucleic Acids Res. 24, 4876-4882 (1997)) and plotting of conservation to molecular surfaces may be performed using the program msf_similarity_to_pdb.

In another embodiment, any of numerous computer programs available and suitable for rational drug design and the processes of computer modeling, model building, and computationally identifying, selecting and evaluating potential inhibitors of gustatory receptor expression, or heterocomplex formation or heterocomplex trafficking, etc., per the methods described herein, may be used, and represent embodiments of this invention. These include, for example, GRID (available form Oxford University, UK), MCSS (available from Molecular Simulations Inc., Burlington, Mass.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Molecular Simulations Inc., Burlington, Mass.), and 3D database systems such as MACCS-3D (available from MDL Information Systems, San Leandro, Calif.), and UNITY (available from Tripos, St. Louis. Mo. Potential agents may also be computationally designed “de novo” using such software packages as LUDI (available from Biosym Technologies, San Diego, Calif.), LEGEND (available from Molecular Simulations Inc., Burlington, Mass.), and LEAPFROG (Tripos Associates, St. Louis, Mo.). Compound deformation energy and electrostatic repulsion, may be evaluated using programs such as GAUSSIAN 92, AMBER, QUANTA/CHARMM, AND INSIGHT II/DISCOVER. These computer evaluation and modeling techniques may be performed on any suitable hardware including for example, workstations available from Silicon Graphics, Sun Microsystems, and the like. Any such use, as pertains to the present invention is to be considered an embodiment thereof. These techniques, methods, hardware and software packages are representative and are not intended to be comprehensive listing, and any such use represents an embodiment of the invention. Other modeling techniques known in the art may also be employed in accordance with this invention. See for example, N. C. Cohen, Molecular Modeling in Drug Design, Academic Press (1996) (and references therein), and software identified at internet sites including the CAOS/CAMM Center Cheminformatics Suite at http://www.caos.kun.nl/, and the NIH Molecular Modeling Home Page at http://www.fi.muni.cz/usr/mejzlik/mirrors/molbio.info.nih.gov/modeling/software list/.

In one embodiment, a screen for sensitivity of neurons to CO₂ can be used to identify agents that interfere with expression, trafficking, or heterodimerization of the gustatory receptor. In one embodiment, electrophysiological measurements are performed in accordance with de Bruyne et al., 2001, Neuron 30, 537-552. In one embodiment, the engineered neurons described above expressing two gustatory receptors can be used.

In another embodiment, the screen may employ the use of a standard calcium FLIPR assay. According to this aspect of the invention, and in one embodiment, Gr21A/Gr63A or a homologue thereof will be co-transfected into standard tissue culture cells and loaded with a calcium sensitive dye, such as fura-2 or calcium-3. Upon CO₂ stimulation, the increase in calcium will be monitored on a FLIPR or FlexStation instrument, capable of reading 384 samples simultaneously (see Marshall et al., Ratiometric Ca2+ measurements using the FlexStation scanning fluorometer, Methods Mol Biol. 2006;312:119-24). In another embodiment, GPRgr22/GPRgr24 or a homologue is used.

In another embodiment, a protein complementation assay to monitor FRET between the two heteromeric subunits may be used, as described in (Remy, I., Galarneau, A., and Michnick, S. W. (2002). Methods Mol Biol 185, 447-459). According to this aspect of the invention, and in one embodiment, two fragments of yellow fluorescent protein (yfp), each comprising roughly one half of the active proteins may be fused to Gr21a and Gr63a, respectively, or a homologue thereof. Both fusion proteins may be co-transfected into cultured cells and fluorescence monitored. If the proteins associate tightly, the fluorescence of yfp will be regenerated and read out by a fluorescent plate reader. Likewise, in another embodiment, GPRgr22 and GPRgr24 or a homologue are used.

Once lead compounds targeted against the Gr21a and Gr63a proteins or their orthologues are identified, they will be tested in Drosophila and Anopheles, respectively, CO₂ sensing behavior and imaging assays as described herein.

In another embodiment, a behavioral assay may be utilized for evaluating the agents of this invention. The behavioral assay may be as described (Suh, G. S., et al. Nature 431, 854-859), where flies carrying Gr21a-Gal4 transgenes, or homologues thereof, with or without UAS-shi^{ts} are tested in a t-maze at permissive (21° C.) or restrictive (28/34° C.) temperatures. Indifference to the odor corresponds to a response index (RI) of 0 while changes in the response, for example, total avoidance in Drosophila will correspond to an RI of −1 or in another embodiment, total attraction in Anopheles will correspond to an RI of +1.

Lead compounds may be optimized by methods well known in the art, and lead and optimized compounds that show promise in cell-based, behavioral, and imaging assays may be subjected to field tests on wild populations of, for example, Anopheles gambiae at sites where vector-borne disease is prevalent, and changes in disease incidence, severity, etc. may be evaluated by standard epidemiologic practice.

In another embodiment, cell-based assay are prepared to identify inhibitors of the CO₂ receptor.

In another embodiment, the various assays described herein can be used to identify agents that function as attractants for insects, for example agents that are as or more potent as attractants than CO₂, for use in baits and other insect traps to lure insects away from the human hosts and towards a trap. Agents that promote expression of the gustatory receptors or formation of heterocomplexes thereof are candidates for such attractants. Agents that increase CO₂ responsivity in ectopically expressed gustatory receptors, in particular in cells amenable to high throughput screening, are embodied herein.

In one embodiment of this invention, inhibiting gustatory receptor expression alters insect responsiveness to carbon dioxide, thereby controlling insect attraction to the subject. In one embodiment, altering insect responsiveness may refer to initiation of a response, or in another embodiment, enhancing a response to carbon dioxide, which in one embodiment, manifests as initiation of attraction to the subject, or in another embodiment, enhanced attraction to the subject, or in another embodiment, directed attraction to the subject.

In another embodiment, altering insect responsiveness may refer to abrogation of a response, or in another embodiment, diminution of a response to carbon dioxide, which in one embodiment, manifests as a lack or absence of attraction to the subject, or in another embodiment, diminished attraction to the subject, or in another embodiment, less directed attraction to the subject.

As described herein, insect responsiveness to CO₂, whether positive or negative may be a function of insect species, or in another embodiment, the amino acid sequence of the gustatory receptor, or in another embodiment, a function thereof. In one embodiment, such preference may specifically be manipulated, via the use of agents, which differ in terms of the stimulatory or inhibitory capacity for insect attraction to a subject, for a particular purpose. For example, and in one embodiment, construction of compositions or netting comprising agents which inhibit or diminish insect attraction to a subject may be used as a means of preventing insect bites in humans. In another embodiment, construction of traps comprising insecticides may be accomplished, wherein an agent which stimulates or enhances insect responsiveness to carbon dioxide is used to attract the insect to the trap, and wherein the trap is constructed to release CO₂, thereby drawing the insect to the trap, where any number of means may be utilized to kill the insect, such as, for example, an insecticide, an electric shock, etc.

In another embodiment of this invention, there is provided an agent, which inhibits gustatory receptor expression or function, as herein described.

It is to be understood that the reference herein to the term “agent” is to include any molecule that performs the indicated function. In one embodiment, the agent is a nucleic acid sequence. In another embodiment, the agent is an expression vector comprising an indicated nucleic acid. In another embodiment, the agent is a peptide, or protein. In another embodiment, the agent is a small molecule. In another embodiment, the agent is derived or isolated from natural sources. In another embodiment, the agent is synthesized. It is to be understood that agent is meant to encompass any of these embodiments, and is meant to include any mediator of an indicated function.

In one embodiment, the small molecule comprises, inter-alia, synthetic organic structures typical of pharmaceuticals, peptides, nucleic acids, peptide nucleic acids, carbohydrates, lipids, and others, as will be appreciated by one skilled in the art. In another embodiment, small molecules may comprise chemically synthesized peptidomimetics.

In one embodiment, according to this aspect of the invention, the agent can be used alone or in combination with other materials in liquid, solid or gaseous form, as part of a composition. In another embodiment, the composition containing the agent may further comprise diluents, extenders, carriers and conditioning agents to provide a composition in the form of finely divided particulate solids, semi-solids, aerosols, solutions, dispersions or emulsions. The composition may comprise any suitable combination, as well. Use of such compositions for effecting the methods of this invention are to be considered as part of this invention, as well.

The composition comprising the agent, according to this aspect of the invention, may also comprise other attractants for the particular insect being controlled, representing another embodiment of the invention. For example, the agent can be applied to or admixed with attractants or baits such as sucrose, glucose, molasses, protein mixtures, powdered egg yolk, powdered milk, yellow corn grits, quincy granules, pumice granules, sex attractants, and the like.

The composition may comprise an insecticide, as described. The term “insecticide” is defined herein to mean a compound, which kills insects. The term may also be understood to include compounds, which reduce fecundity. Numerous such compounds are known in the art, particularly pyrethroid, carbamate and organophosphate insecticides.

Numerous devices for the delivery of such agents to insects are well known in the art (see for example, U.S. Pat. No. 4,666,767 or U.S. Pat. No. 4,639,393), and their implementation with agents and compositions of the invention, as described herein, represent additional embodiments of the invention.

Nylon bed nets impregnated with insecticides have been used to reduce the incidence of insect-borne diseases. In one embodiment of this invention, a nylon bed net is envisioned, which is impregnated with an agent as described herein, and may further comprise other compounds, such as insecticides, or other repellents.

In another embodiment, this invention provides a method of controlling insect attraction to a subject, where the method comprises the step of inhibiting formation of a heterocomplex of gustatory receptors in said insect, whereby inhibiting said formation of said heterocomplex alters said insect responsiveness to carbon dioxide, thereby controlling insect attraction to said subject.

As used herein, the term “exposing”, “exposure” or “exposed” refers to direct and indirect exposure to a nucleic acid, peptide, protein, complex, vector, agent or composition, as described herein. In one embodiment, exposure may be proximal exposure by an insect to a substrate comprising an agent, composition, etc., as herein described, for example, when the agent is used to coat a netting.

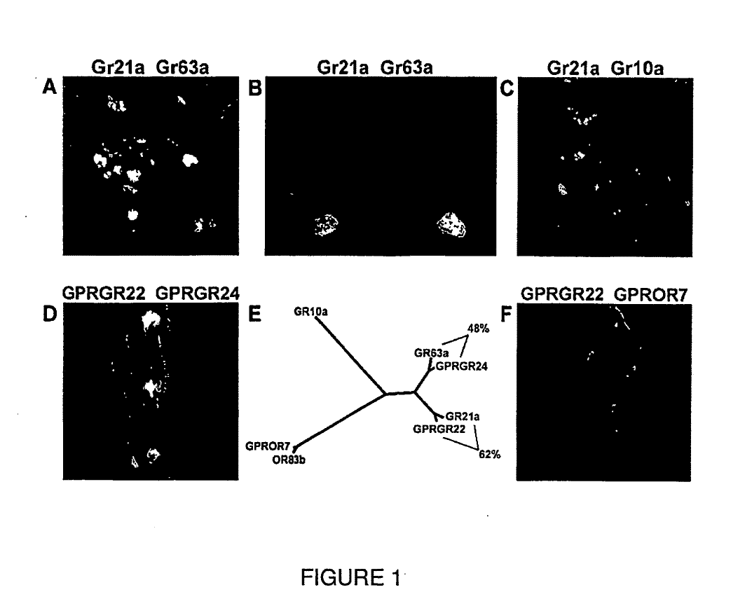

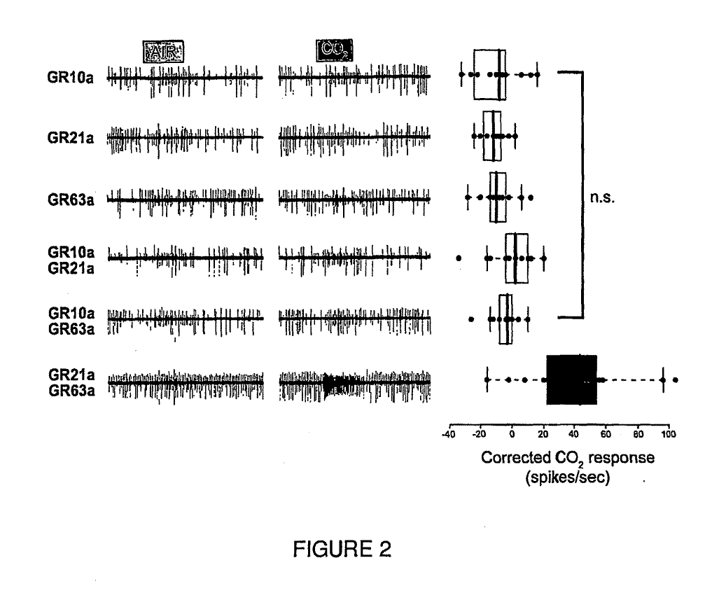

As shown in FIG. 1, Gr21a is co-expressed with Gr63a in ab1C neurons and neurons expressing the two converge upon the same target in the antennal lobe. Single sensillum recordings for control insects versus those expressing Gr21a/Gr63a were shown herein to provide specific responses to CO₂ only in animals expressing Gr21a and Gr63a (FIG. 2). Thus, in one embodiment of this invention, CO₂ responsiveness involves heterocomplex formation between Gr21a and Gr63a, or homologues thereof, and affecting CO₂ responsiveness according to the methods of this invention, in some embodiments, is via interfering with the heterocomplex interaction. In one embodiment, inhibiting heterocomplex interaction inhibits insect attraction to a subject, as described.

Likewise, co-expression of the CO₂ receptor subunits is found in the malaria mosquito, Anopheles gambiae. The Gr21a and Gr63a orthologues, GPRgr22 and GPRgr24, are co-expressed in the CO₂-sensitive olfactory organ in the mosquito, the maxillary palp (FIG. 1D). As in Drosophila, the putative CO₂ receptor is not co-expressed with the ubiquitous olfactory co-receptor of the OR83b subtype (GPROR7 in the mosquito) (FIG. 1F).

In one embodiment, inhibiting gustatory receptor heterocomplex formation is accomplished via the use of an agent, which may represent any embodiment as described herein for what is encompassed by the term “agent”. In one embodiment, the agent may be identified via any number of means including rational design, or random design, as described herein, and may be validated, via functional analysis in terms of inhibiting heterocomplex formation, insect attraction, insect responsiveness to carbon dioxide, as will be known to one skilled in the art, and as described and exemplified herein.

In another embodiment, this invention provides a method of inhibiting, preventing or reducing the incidence of insect-borne disease in a subject, the method comprising inhibiting gustatory receptor expression in said insect, whereby inhibiting said expression alters said insect responsiveness to carbon dioxide, controlling insect attraction to said subject, thereby inhibiting, preventing or reducing the incidence of insect-borne disease in a subject.

In another embodiment, this invention provides a method of inhibiting, preventing or reducing the incidence of insect-borne disease in a subject, the method comprising inhibiting formation of a heterocomplex of gustatory receptors in said insect, whereby inhibiting formation of said heterocomplex alters insect responsiveness to carbon dioxide and attraction to said subject, thereby inhibiting, preventing or reducing the incidence of insect-borne disease in a subject.

It is to be understood that any embodiment as described herein for agents and means of inhibiting gustatory receptor expression or heterocomplex formation may be used for methods of preventing insect borne disease and are to be considered as part of this invention.

In another embodiment, this invention provides an agent, which inhibits gustatory receptor heterocomplex formation or trafficking of said complex to ciliated dendrites.

In one embodiment, the insect-borne disease is of humans, or in another embodiment, other animals. In some embodiments, the insect-borne disease comprises those carried by mosquitoes, which may be vectors for malaria, yellow fever, dengue fever, and West Nile encephalitis, Rift Valley fever, arboviral encephalitides, such as eastern equine encephalitis, Japanese encephalitis, La Crosse encephalitis, St. Louis encephalitis, West Nile virus and western equine encephalitis, and filariasis. In some embodiments, the insect-borne disease comprises those carried by ticks, which may be vectors for babesiosis, ehrlichiosis, Lyme disease, Rocky Mountain spotted fever, southern tick-associated rash illness, tick typhus, tularemia and encephalitis. In some embodiments, the insect-borne disease comprises those carried by sand fleas, which may be vectors for leishmaniasis, Carrion's disease and sand fly fever. In some embodiments, the insect-borne disease comprises those carried by tsetse flies, which may be vectors for African sleeping sickness. In some embodiments, the insect-borne disease comprises those carried by assassin bugs, which may be vectors for Chagas' disease. In some embodiments, the insect-borne disease comprises those carried by lice, which may be vectors for lice infestation, epidemic relapsing fever, trench fever and typhus fever. In some embodiments, the insect-borne disease comprises those carried by black flies, which may be vectors for filariasis and onchocerciasis. In some embodiments, the insect-borne disease comprises those carried by horse flies and deer flies, which may be vectors for tularemia, anthrax and loaiasis. In some embodiments, the insect-borne disease comprises those carried by eye gnats, which may be vectors for yaws and conjunctivitis. In some embodiments, the insect-borne disease comprises those carried by house flies, which may be vectors for dysentery, typhoid fever, cholera and poliomyelitis. In some embodiments, the insect-borne disease comprises those carried by fleas, which may be carriers of bubonic plague and murine typhus. In addition, various parasitic, rickettsial, bacterial and viral diseases of animals and man are spread by mosquitoes, ticks, biting flies, fleas, lice and other biting insects, any of which may be affected or prevented by the methods of this invention, or using agents of this invention. Application of the agents or compositions of the invention, or accordance with the methods of this invention, in one embodiment, reduces the incidence of the diseases in humans and animals by reducing the number of insect bites. The term insect bites as used herein includes methods of transmission where the disease is not necessarily transmitted from the insect to the host directly through the mouthparts of the insect, but may be transmitted in association with the bite wound. For example, parasites in the feces of the insect may be deposited on the hosts's skin after feeding and parasites enter the host via the wound by proximity or scratching, as occurs in the transmission of Chagas' disease, or in the case of mosquito-transmitted filariasis, in which the parasites exit the insect's body onto the host while the insect is feeding. “Bites” thus embraces all methods of disease transmission associated with the attraction of an infected insect onto or near the skin of the host.

In one embodiment, the methods of this invention may be carried out via topical application of an agent inhibiting gustatory receptor expression or heterocomplex formation to the subject. The agent, in one embodiment, may be applied as part of a composition, as described, which may also contain carriers, emulsifiers, or diluents as known to one of ordinary skill in the art. The specific carrier used in a composition of or for use in this invention, depends on how the repellent composition will be applied (whether in a lotion, spray or dust form, for example) and where the repellent composition will be applied.

Spray formulations are known to one of ordinary skill in the art and include aqueous solutions, water-soluble powders, emulsifiable concentrates, water miscible liquids/powders (for compositions that are soluble in water), wettable powders or water-dispersible powders, flowable/sprayable suspensions or suspension concentrates, and oil solutions.

Emulsifiers useful in the invention are generally known to one of ordinary skill in the art and include, but are not limited to the following: non-ionic or ionic polymers such as polyoxyethylenesorbitan monooleates (Tweens), such as Tween 20 and Tween 60; sorbitol (polysorbate 80); propylene glycol; polyethylene glycol; ethanol (ethyl alcohol); and methanol (methyl alcohol). Other surfactants that can be used as an emulsifier for pesticide formulations are the phosphate esters. Examples of commercially available phosphate ester surfactants include, but are not limited to the following: butyl phosphate; hexyl phosphate; 2-ethylhexyl phosphate; octyl phosphate; decyl phosphate; octyldecyl phosphate; mixed alkyl phosphate; hexyl polyphosphate; and octyl polyphosphate. Preferably, the emulsifier used is either plant extract, bees wax or commercial emsulfiers/surfactants such as Novomer EC-1, Ultrez 21, Pemulen TR-2NF, soaps, poloxamers, Tweens and the like). More preferably, a plant extract (e.g., plant lecithin, plant glycerin, plant waxes and glycoproteins) is used as the emulsifier if an emulsifiable concentrate of a repellent composition of the invention is to be formulated.

In one embodiment, assessment of the inhibition of insect responsiveness to CO₂ may be conducted via any means known in the art, some of which are exemplified herein. In one embodiment, such assessment may comprise the WHO test protocol for mosquito repellents (Barnard, D. R. 2000. Repellent and toxicants for personal protection. WHO/CDS/WHOPES). According to this aspect of the invention, and in one embodiment, clear Perspex™ mosquito cages (38×38×38 cm) containing more than 400 adults of both sexes and various ages are used. The compositions comprising agents are introduced to forearms of a subject, up to the elbow, through a cloth sleeve entry port into the cage. An untreated arm, or one treated with vehicle alone is exposed for a 3-minute period. The number of landings and bites are recorded as bites per 3-minute period. Subsequently each forearm is treated with agents/compositions as described herein, and introduced into the cages for a further 3 minutes of evaluation. Results may also be obtained for forearm exposure over a course of time.

### Examples

Materials and Methods. Drosophila strains. The following transgenic Drosophila strains were used: 1) Gr63a-syntRFP, which expresses a Gr63a-synthetic red fluorescent protein fusion product to Drosophila synaptotagmin: 3 kilobases immediately upstream of the GR63a translation initiation codon were cloned upstream of a translational fusion of Drosophila synaptotagmin to red fluorescent protein. The resulting fragment was cloned into standard Drosophila P-element vector Casper and transgenic flies generated by standard methods; 2) Gr21a-GAL4, which expresses a Gr21a-promoter driving expression of the yeast Gal4 transcription factor (Scott, K., et al. (2001). Cell 104, 661-673); and 3) UAS-CD8-GFP (Lee, T., and Luo, L. (1999). Neuron 22, 451-461).

In addition, flies carrying Gr21a-Gal4 (Scott, K., et al. (2001). Cell 104, 661-673) transgenes with or without UAS-shi^{ts} were constructed (Dubnau, J., Grady, L., Kitamoto, T., and Tully, T. (2001). Disruption of neurotransmission in Drosophila mushroom body blocks retrieval but not acquisition of memory. Nature 411, 476-480)

Colocalization Studies, In situ Hybridization. Fluorescent double in situ hybridization to Drosophila Gr21a, and Gr63a or Gr10a was conducted as described (Vosshall, L. B., et al. (2000). Cell 102, 147-159. Gr21a was probed with a synthetic mRNA fragment specifically recognizing Gr21a, conjugated to FITC probe, and Gr63a or Gr10a probed with synthetic mRNAs conjugated to digoxigenin (DIG). Sequential TSA amplifications using TSA-FITC and TSA-Cy5 kits (Perkin Elmer) were conducted, with peroxidase quenching between reactions to allow for visualization of weak signals.

Colocalization Studies, Fluorescence Microscopy. Transgenic fly brains were dissected and labeled with an antibody recognizing central brain neuropil (nc82) [Laissue, P. P. et al. J. Comp. Neurol. 405, 543-552 (1999)] and probed with a secondary antibody coupled to Cy5 and visualized by confocal fluorescence microscopy using a Zeiss LSM-510 laser scanning confocal microscope.

CO₂ Sensation. Non-ab1C and ab1C neuron response to CO₂ was assessed as described (de Bruyne, M., Foster, K., and Carlson, J. R. (2001). Neuron 30, 537-552), following exposure for roughly 25 seconds to 1% CO₂ stimulation.

ab3A neurons were engineered to ectopically express the following gustatory receptor(s): Gr10a, Gr21a, Gr63a, Gr10a+Gr21a, Gr10a+Gr63a, and Gr21a+Gr63a.

Generation of Gr63a null mutant flies. Homologous recombination was carried out as described [Larsson, L., Domingos, A. I., Jones, W. D., Chiappe, M. E., Amrein, H. & Vosshall, L. B. Or83b encodes a broadly expressed odorant receptor essential for Drosophila olfaction. Neuron. 43:703-714 (2004)] using an end-out targeting vector that flanked the Gr63a coding region. Descendants of flies carrying the eye color marker of this targeting vector on the third chromosome were assessed genetically and by physical mapping of the deletion by PCR.

### Example 1

### Gr21a and Gr63a are Coexpressed in ab1C Neurons

The ab1C class neurons in the adult antenna have been identified as the only sensory neurons in the fly that are responsive to CO₂. Gr21a is a marker for the ab1C neurons. In order to determine whether the Gr21a receptor mediates the detection of CO₂, it was ectopically expressed in a large number of olfactory sensory neurons (OSNs) that normally do not respond to CO₂, and whether this receptor alone could confer CO₂ sensitivity was assayed.

When GFP-tagged Gr21a was ectopically expressed in OSNs, which expressed members of the class of general odorant receptors (ORs) and the universal co-receptor Or83b, Gr21a did not traffic to ciliated OSN dendrites [Benton, R., Sachse, S., Michnick, S. W. & Vosshall, L. B. Atypical membrane topology and heteromeric function of Drosophila odorant receptors in vivo. PLoS Biol. 4:e20 (2006).]. Since a requirement for heterodimerization of GRs with Or83b exists, to support OR/OR83b trafficking and function in ciliated OSN dendrites, it was of interest to determine whether a second GR gene co-expressed in the antenna along with Gr21a may similarly mediate trafficking to the OSN dendrites.

In a detailed expression analysis of the GR gene family, three GRs were detected in the antenna, Gr21a, Gr63a, and Gr10a, and Gr63a, and Gr10a co-expression with Gr21a was determined. Toward this end, two-color fluorescent in situ hybridization was conducted, and Gr21a co-expression with Gr63a (FIG. 1A) but not with Gr10a (FIG. 1C) was found in ab1C neurons.

In order to confirm colocalization, neuronal expression of Gr21a-specific and Gr63a-specific transgenic marker convergence upon the same target in the antennal lobe was determined. Indeed, both transgenes specific for Gr21a and Gr63a label the same population of OSNs that synapses in the V glomerulus, the sole antennal lobe region that responds to CO₂ (FIG. 1B).

### Example 2

### Gr21a and Gr63a Coexpression Mediates Trafficking to OSN Dendrites

Given that Gr21a and Gr63a are co-expressed in vivo, it was of interest to determine whether expressing both receptors ectopically would permit proper trafficking of the heteromeric receptor complex to OSN dendrites. When GFP:Gr21a and Gr63a are ectopically expressed under the control of the Or83b-Gal4 driver, small but reproducible amounts of GFP:Gr21a were detected in the ciliated dendrites.

### Example 3

### Gr21a and Gr63a Complexes Mediate CO₂ Sensation

To determine whether these receptor complexes are functional in the cilia, single sensillum recordings were performed. Different various single GRs and combinations of GRs were ectopically expressed in a non-CO₂ neuron, ab3A, a sensillum normally responsive to ethyl butyrate. Of neurons expressing either Gr10a, Gr21a, Gr63a, Gr10a+Gr21a, Gr10a+Gr63a, or Gr21a+Gr63a (FIG. 2), only the combination of Gr21a with Gr63a confers sensitivity to CO₂ (FIG. 2, last row). Sample traces show 4 seconds of activity surrounding a 1 second stimulus of either room air (0.03% CO₂) or roughly 3% CO₂. Spikes were counted in a 500 ms window immediately following the stimulus. 2(CO₂−Air) gives a corrected CO₂ response in spikes/sec. N=12-18 sensilla/genotype. This gain-of-function experiment demonstrates that both subunits are necessary to reconstitute a CO₂ receptor in vivo.

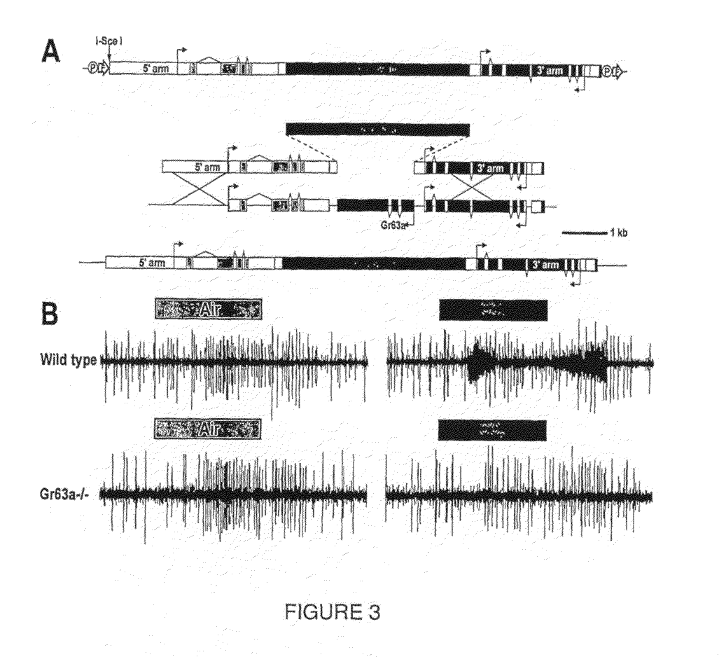

In addition, Drosophila which are mutated for Gr63a were constructed by homologous recombination (FIG. 3A), with the targeting construct (top), schematic of site of homologous recombination (middle), and mutant chromosome (bottom). The flanking genes included in the targeting construct are not disrupted by this manipulation. As shown in FIG. 3B, these mutant flies fail to respond to CO₂ electrophysiologically. Representative traces are shown from wild type (top) and Gr63a−/− (bottom) ab1 sensilla. Sample traces show 2.5 sec of activity with a 1 sec pulse of 1 second stimulus of either room air (0.03% CO₂) or roughly 3% CO₂. Note that spontaneous activity is unaffected in the mutant, but that there are no CO₂-evoked spikes in Gr63a−/− mutants. These data show that Gr63a is an essential component of a CO₂ receptor in Drosophila.

### Example 4

### Expression of GPRgr21 and GPRgr24 in Anopheles

GPRgr22 and GPRgr24 are the mosquito orthologues of the Drosophila CO₂ receptor subunits Gr21a and G63a, showing 62% and 48% identity (FIG. 1E). GPRgr22 and GPRgr24 are co-expressed in the CO₂-sensitive olfactory organ, the maxillary palp, in the mosquito Anopheles gambiae (FIG. 1D). As in Drosophila, the putative CO₂ receptor is not co-expressed with the ubiquitous olfactory co-receptor of the OR83b subtype (GPRor7 in the mosquito) (FIG. 1F).

### Example 5

### Gr21a and Gr63a Mediation of Behavioral Responses to CO₂

Fruit flies and mosquitoes both show strong, but very different behavioral responses to CO₂. CO₂ is the most potent repellent known for fruit flies, while the same stimulus is a potent attractant for mosquitoes. Since blockade of Gr21a accomplished via methods as described (Suh, G. S., et al. (2004). Nature 431, 854-859) showed that such blockade in Drosophila prevents repulsion to CO₂, Gr63a may be similarly evaluated.

In order to determine whether the Gr21a and Gr63a orthologues (GPRgr22 and GPRgr24, respectively) will affect mosquito attraction to CO₂, comparable constructs with the respective genes will be introduced into mosquitoes, and their behavioral response will be similarly evaluated.

RNA interference may be used to “knock down” expression, to evaluate whether Anopheles or other orthologues of the Drosophila CO₂ receptors are behaviorally necessary for CO₂-evoked behavior, assessed as described in this example, in one embodiment of this invention. Imaging techniques may also be used for these determinations, for example as described hereinabove.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-38. (canceled)

39. A method comprising the step of inhibiting an expression of a gustatory receptor in an insect with an agent, wherein said inhibiting alters a response of said insect to carbon dioxide.

40. The method of claim 39, wherein said inhibiting controls an attraction of said insect to a subject.

41. The method of claim 40 wherein said controlling inhibits, prevents or reduces the incidence of an insect-borne disease in said subject.

42. The method of claim 39 wherein said gustatory receptor is selected from the group consisting of Gr21a, Gr63a, a homologue of Gr21a, a homologue of Gr63a, GPRgr22, GPRgr24, a homologue of GPRgr22, and a homologue of GPRgr24.

43. The method of claim 39 wherein said agent is selected from the group consisting of a nucleic acid and a small molecule.

44. The method of claim 39 wherein said agent is applied to a netting.

45. The method of claim 41 wherein said disease is selected from the group consisting of malaria, dengue, yellow fever, river blindness, lymphatic filiariasis, sleeping sickness, leishmaniasis, epidemic polyarthritis, West Nile virus disease and Australian encephalitis.

46. A method comprising the step of inhibiting a formation of a heterocomplex of gustatory receptors in an insect with an agent, wherein said inhibiting alters a response of said insect to carbon dioxide.

47. The method of claim 46, wherein said inhibiting controls an attraction of said insect to a subject.

48. The method of claim 47 wherein said controlling inhibits, prevents or reduces the incidence of an insect-borne disease in said subject.

49. The method of claim 46 wherein said heterocomplex of gustatory receptors is selected from the group consisting of Gr21a, Gr63a, a homologue of Gr21a, a homologue of Gr63a, GPRgr22, GPRgr24, a homologue of GPRgr22, and a homologue of GPRgr24.

50. The method of claim 46 wherein an exposure of said insect to said agent inhibits said formation.

51. The method of claim 46 wherein said agent is applied to a netting.

52. The method of claim 48 wherein said disease is selected from the group consisting of malaria, dengue, yellow fever, river blindness, lymphatic filiariasis, sleeping sickness, leishmaniasis, epidemic polyarthritis, West Nile virus disease and Australian encephalitis.

53. An isolated protein complex comprising insect gustatory receptors selected from the group consisting of Gr21a, Gr63a, a homologue of Gr21a, a homologue of Gr63a, GPRgr22, GPRgr24, a homologue of GPRgr22, and a homologue of GPRgr24.

54. A modified neuron engineered to express a gustatory receptor selected from the group consisting of Gr21a, Gr63a, a homologue of Gr21a, a homologue of Gr63a, GPRgr22, GPRgr24, a homologue of GPRgr22, and a homologue of GPRgr24.

55. The modified neuron of claim 54 wherein said neuron is an ab3A neuron.

56. A Drosophila mutant wherein said mutant is null with respect to Gr21a or Gr63a.


SEQUENCE LISTING (4 sequences)

SEQ ID NO: 1 - protein, 467 aa, Anopheles gambiae
MIHTQMEDAQYEIRHQVLNPNQRQQLEDRRRIKEQLHQLEQDNESPTHMYRRKLKIASDVNLLDQHDSFYHTTKSLLVLFQIMGVMPIMRSPKGVDMPRTTFTWCSKAFLWAYFIYACETVIVLVVARERINKFISTSDKRFDEVIYNIIFMSIMVPHFLLPVASWRNGSEVAKFKNMWTDFQYKYLIVTGKPIVFPKLYPITWTLCIVSWSLSLVIILSQYYLQPDFQFCHTFAYYHIIAMLNGFCSLWFVNCTAFGTASKAFAKELTDVLATERPAAKLTEYRHLWVDLSHMMQQLGKAYSNMYGIYCLVIFFTTIIATYGSLSEIIEHGATYKEVGLFVIVFYCMSLLFIICNEAHHASKRVGLNFQERLLNVNLTAVDKATQKEVEMFLVAIDKNPPTMNLDGYANINRGLITSNISFMATYLVVLMQFKLTLLRQSAKNAFISALKANLSRIRSLDADKVNT

SEQ ID NO: 2 - DNA, 1404 bp, Anopheles gambiae
atgattcacacacagatggaagatgctcagtacgagattcgccatcaggtgctgaatccgaatcaacgtcagcagctggaggacaggcgccgcatcaaggagcaactgcatcagctcgaacaggataacgagtctccgacgcacatgtacaggcgtaagctgaagattgctagcgatgtaaacctgctcgaccagcacgattcgttctaccacaccaccaagagtctgctggtgctgttccaaattatgggcgtgatgccgataatgcgcagcccgaaaggtgtcgacatgccccgtaccacgtttacctggtgctccaaggcgtttctctgggcgtactttatctacgcctgcgaaacggtcattgtgctggtggtggcccgggagcgcattaacaaattcatctcaaccagcgacaagcggttcgatgtaatggtgcccttcccgtagaattcaaaaagattttcagttcgtttttccccgatcacgtgtttggtgataggtgatctacaacatcatctttatgtcgaccattttctgcaagctggcgtaacggttcggaggtggcaacatgtggacaacaagtatctcatcgtcaccggaaagcccagaagctgtatggaccttgtgcatcgtatcgtggtcgttgacatcctgtcgcaatattacctgcagccagactttcagttctgtcacacctttgcctactaccacatcattgcaatgttgaacgggttctgtagcctttggttcgtcaactgcaccgcatttgggacggctagtaaggcctttgccaaagaactaacagacgtactggcgaccgaacggccagccgcgaaactgaccgaatatcgtcacctgtgggtggatttaagccatatgatgcaacaactgggtaaagcatattccaacatgtacggcatctactgtttggtgatattcttcaccactattatcgccacctacggctcgctgagcgaaattatcgagcacggtgccacgtataaggaagtgggtttatttgtcattgttttctattgtatgagcttgctgttcatcatttgcaacgaagctcatcacgcatccaaaagggtcggattgaactttcaggaacgtttgttgaatgtaaatttaacggcggtggacaaggctacacaaaaggaggtagaaatgtttctggttgccatcgacaaaaatccccctacgatgaatcttgacgggtacgccaacatcaatcgcggactcatcacatcgaacatatccttcatggcgacgtatttggtcgttttgatgcagttcaaattgaccttactacgacaaagtgctaaaaatgctttcatttctgcactcaaggctaacttgtctagaattcgatcgcttgacgcagacaaagtgaacacctaa

SEQ ID NO: 3 - protein, 378 aa, Anopheles gambiae
MVFESSKPIYLVLRAIGVLPYTRLPSGGTAFVLASPSMTYCVLFFLLLTVYIAFILLNRIEIVRTLEGRFEESVIAYLFIVNILPILIIPLMWYESRKVVSVVNGWVDFETVYRETTGRALELRLRTKAQVIAILLPILCSLSVAITHVTMVDFKLLQVIPYCVLDTITYMMGGYWYMACETLSITAKILAEDFQRALRHVGPAAKVSEYRSLWLRLSKLARDTGFSTCYTFTFICLYLFFIITLSIYGLMSQISDGFGVKDIGLAVTAFCSVGLLFYICDEAHYASFNVRTNFQKKLLMVELSWMNTDAQTEINMFLRATEMNPSSINLGGFFDVNRTLFKSLLATMVTYLVVLLQFQISIPDEPSAMLMHSNSSHS

SEQ ID NO: 4 - DNA, 1137 bp, Anopheles gambiae
atggtgtttgaaagctccaaaccgatctacctggtgctgcgtgccatcggtgtgctgccgtacacgcggctcccgtcgggcggaaccgcttttgtccttgcctccccgtcgatgacgtactgtgtgctgtttttcctgctgctcaccgtgtacattgccttcatcctgctcaatcgcatcgagatcgtgcgcacgctcgagggccgcttcgaggagtccgtcattgcgtacctgttcatcgtgaacattctgccgatcctgatcataccgctgatgtggtacgaatcgcgcaaggtcgtgtcggtcgtgaatgggtgggtcgattttgaaaccgtctaccgggaaacaacgggccgggcgctggagttgcgcctgcgcaccaaagcgcaggtcatagcgatcctgctgccgatcctgtgctccctctcggtcgccatcacgcacgtgacgatggtggacttcaagctgctgcaggtcatcccgtactgtgtgctcgacacgatcacctacatgatgggcgggtactggtatatggcctgtgagacgctcagcattaccgcgaaaatactggcggaagactttcagcgcgcgctacggcacgtcggcccggcggcgaaggtgtccgagtatcggtcgctatggttacggctgagcaagctggcacgggatacgggcttttccacctgctacacgttcacgttcatctgtctgtatctgttcttcatcataacgctttcgatctatgggctgatgtcgcaaatttcggacggcttcggcgtgaaggacatcgggctggcggtgaccgccttctgtagcgtggggctgctgttctacatctgcgacgaggcgcactacgcgtcgttcaatgtgcgcaccaacttccagaagaagctgctgatggtggagctgagctggatgaacacggacgcgcagacggagattaatatgtttctgcgtgcgaccgagatgaatccttccagcatcaatcttggcgggtttttcgacgttaatcgcacgctgtttaagtcgctgcttgccacgatggttacgtatctggtggtgttgctacagttccagatcagtattcccgatgagcccagcgccatgctgatgcacagtaattcgtctcattcttag